(19) United States
(12) Patent Application Publication
(10) Pub. No.: US 2007/0083096 A1
(43) Pub. Date: Apr. 12, 2007

(54) Knitted textile for the monitoring of vital signals

(76) Inventor: Rita Paradiso, Guano-Capannori (IT)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 10/581,476

(22) PCT Filed: Dec. 3, 2004
(86) PCT No.: PCT/EP04/53251

§ 371 Date: Jun. 1, 2006

(30) Foreign Application Priority Data
Dec. 3, 2003 (IT) FI2003A000308

(51) Int. Cl. A61B 5/04

(52) U.S. Cl. 600/388

(57) ABSTRACT

A non-invasive monitoring and, in particular, a wearable system for the monitoring of vital signals (Cardiovascular, respiratory and an index of movement) to be used for the detection of potential pathologies via the acquisition, elaboration and transmission of signals, where the sensitive elements are done with knitted fabric, in direct contact with the body and that could be, if necessary, elastic, where yarns, having characteristics that allows the production of fabric piezoresistive regions and fabric conductive regions working as electrodes or connections, are integrated.

### FIELD OF INVENTION

The present invention relates the non-invasive monitoring of cardio-respiratory functions

### BACKGROUND OF THE INVENTION

Several types of apparatus exist, that are used in the field of medicine, for monitoring heart functionality of at-risk patients, such as post-infarcted patients or people with various cardiac pathologies, and people that occasionally or regularly carry out actions with a high risk for their heart, such as sport activities—as professional or amateur athletes—or heavy or dangerous jobs, as fire-fighters, soldiers, high-specialised personnel that for safety reasons wears heavy protective clothes and works in extreme conditions, and more in general people carrying out operations that for extreme conditions or for the required level of attention, are exposed to distress and to a consequent modification of their own physiological functionalities.

In hospitals heart functionality of patients are usually controlled by the analysis of the electrocardiogram (ECG), which is a graph of the cardiac potential versus time. The ECG is the most important clinical investigation in cardiac diagnosis. Thanks to information provided by ECG, it is possible to detect the presence of alterations of cardiac rhythm, alterations in the propagation of electric impulses (conduction alterations) or myocardial alterations as a consequence of an ischemia (coronary diseases).

The analysis of variability of cardiac frequency can be done in the frequency domain, by the spectral analysis of signals and the recognition and relative comparison of principal oscillating components, or in the time domain., which is based instead on the measurement of the cardiac cycle and on its beat-to-beat variability measured in milliseconds.

The analysis, both in the frequency and in the time domains, of the cardiac frequency is fundamental. In fact, in patients suffering with Congestive Heart Failure (CHF), cardiac frequency is surely a rough parameter of the autonomic activity of the heart. In these patients an increase in frequency during day time (mainly due to an increased sympathetic tone) and a missing slowdown of the cardiac frequency during night time (expression of an altered vagal control) is observed. It is likely that all cardiovascular diseases are associated to specific changes of autonomic control of the cardiovascular system, which rule the pathogenesis, the development, the evolution of the clinical description and the potential occurring of complications.

This is the reason why today several techniques are under study to determine complex interactions between the Autonomic Nervous System and the Cardiovascular System.

Monitoring of professional sportsmen/women and workers foresees also the correlation of the cardiac data with the respiratory activity, the control of oxygen saturation level and the monitoring of movement activity.

Respiratory activity can be studied recording the changes of the abdominal wall and the thoracic wall associated with inspiration and expiration of the same respiratory cycle.

The correlation of the cardiac signal with the respiratory signal gives further information in respect to those coming from the analysis of a single signal, and specifically indexes correlated to patient's sympathovagal activity can be obtained, activity that is controlled by the Autonomic Nervous System through its two components, sympathetic and parasympathetic, reacts to all changes in the activity of the cardiovascular apparatus, counteracting in a way to maintain homeostasis of the whole system.

The analysis of cardiac frequency variability and respiratory rhythm gives information not only on the balance of activity of Sympathetic and Parasympathetic Nervous Systems, but also hints on risk of heart arrhythmias and failure.

With respect to standard ambulatory heart monitoring techniques (Holter), the proposed system can:

use a device for the monitoring that is really wearable, where user interface is realised with knitting techniques and is made itself with fabric;

integrate and correlate physiological signals, e.g. electrocardiogram, electromyogram, breath, movement;

place electrodes and sensors on the user automatically, as sensitive elements are part of the garment;

monitor continuously user's state.

Other systems based on the same philosophy (Vivometrics), are realised inserting, inside a waistcoat or a jacket, electrodes, conductive yarns, standard sensors that are embedded in the garment, and none of the system that are now the State-of-the-Art uses sensors that are integrated in the garment. Furthermore, non of these systems is able to record an electrocardiogram in continuous.

### SUMMARY

The present invention refers to a knitted fabric where piezoresistive sensors for the monitoring of movement and thoracic and abdominal breathing, electrodes for the monitoring of cardiac activity and for the monitoring of respiratory activity using impedenzometry techniques, and conductive connections for the transmission of signals are integrated.

### BRIEF DESCRIPTION OF DRAWINGS

FIGS. 1 and 2 (a) (b) show the position of sensors with respect to the body of the user (front, side and back view, respectively)

FIG. 3 shows an example of the connection between textile tracks and the electronic device for signal acquisition, for the detection of the signal coming from the electrodes

FIG. 4 shows schematically courses that form the weft knitting

FIG. 5 (a-c) show three different techniques of knitting, double-bed jersey knitted fabric (5a), sensor intarsia (5b), and connection insulation (5c), respectively.

FIG. 6 shows the position of sensors used for impedance pneumography with respect to the body of the user (front view).

### DETAILED DESCRIPTION OF THE INVENTION

The present invention refers to a knitted fabric where piezoresistive sensors for the monitoring of movement and thoracic and abdominal breathing, electrodes for the monitoring of cardiac activity and breathing, and conductive connections for the transmission of signals are integrated, and signals are transferred on hardware able to elaborate acquired data in real time and to transmit to a remote system, which is available to external operators connected to a network of users realised, for instance, via internet or mobile telephone providers, or simply delivered to a PC, where they are visualised, stored and, if necessary, elaborated.

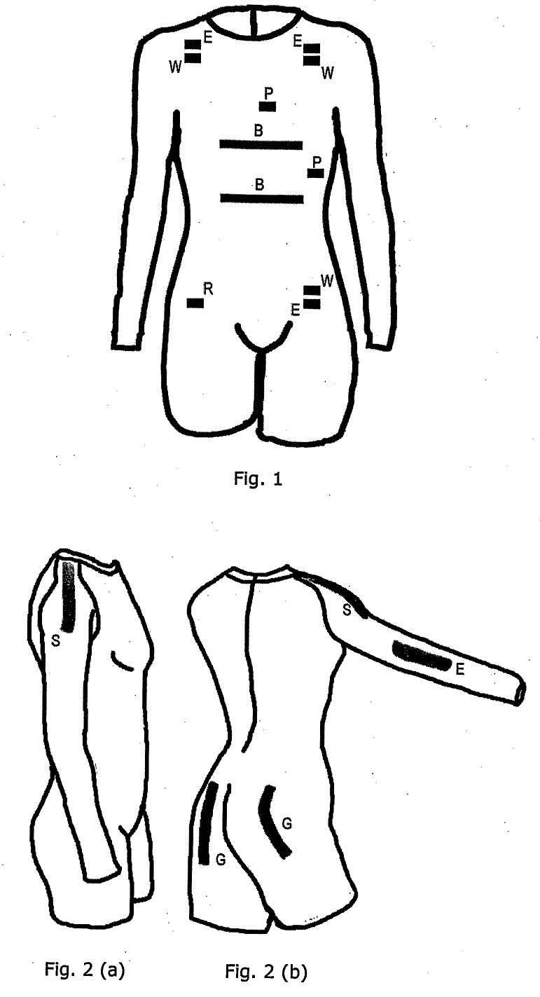

With the use of the present invention, as explained above, it is possible to detect the electrocardiogram with five (5) leads (with reference to FIG. 1, position of electrodes is shown: Einthoven configuration (E), Wilson configuration (W), precordial configuration (P), and reference electrodes (R)), the respiratory frequency (with reference to FIG. 1, position of piezoresistive sensors (B) is shown) and to monitor movement activity for the patient under examination (through the use of piezoresistive sensors S, G and E as shown in FIG. 2). Furthermore, above-mentioned electrodes are able to take the Electrooculogram (EOG) and Electromyogram (EMG).

According to a preferred embodiment of the present invention the knitted fabric is made with the double-bed jersey technique. In the regions where electrodes and connections (that links sensors to the portable electronic device) are located, multiple fabric layers, preferably two or three fabric layers, are present; electrodes are obviously in contact with the skin of the user under examination while connections are insulated by a layer of fabric which separates them from the user's body. Piezoresistive sensors are integrated into fabric and are made of regions made of piezoresistive yarns instead of ground yarns. Said electrodes, connections and sensors are made using a well-known knitting technique, known as “intarsia”. Electrodes and connections are made using a well-known knitting technique, the so-called “tubular intarsia technique” where conductive yarns are used to make sensorised and connective regions.

The knitting tubular intarsia technique allows the realization of separated regions using different yarns, these regions can be linked using connections as described in FIG. 3 where sensors 32, connectors 30 and the electronic device 31 for collection of taken signals are shown.

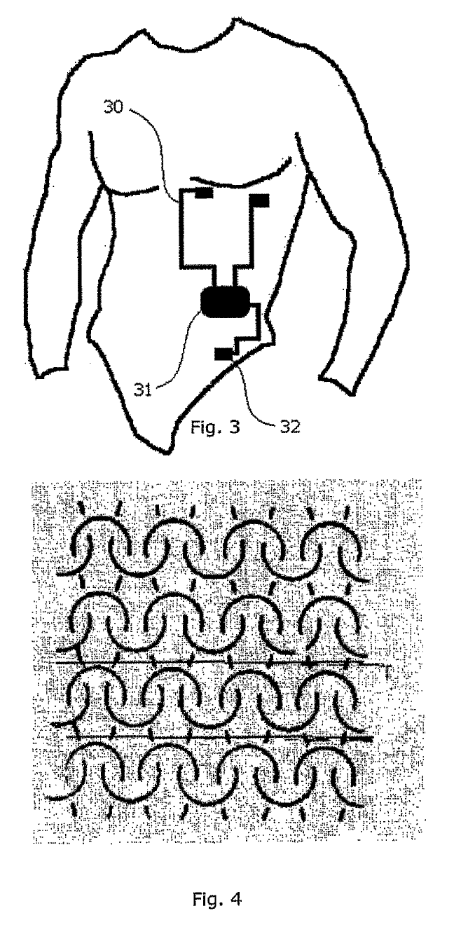

For this type of fabric production, double-bed weft knitting machines are used, intarsia are made by using in the same course (a horizontal row of loops, as shown in FIG. 4), two or more yarn-carriers with guides limited in length and lightly overlapping; each yarn-carrier corresponds to a type of yarn, and in the described system there are two different yarns: ground yarn and conductive yarn, or ground yarn and piezoresistive yarn.

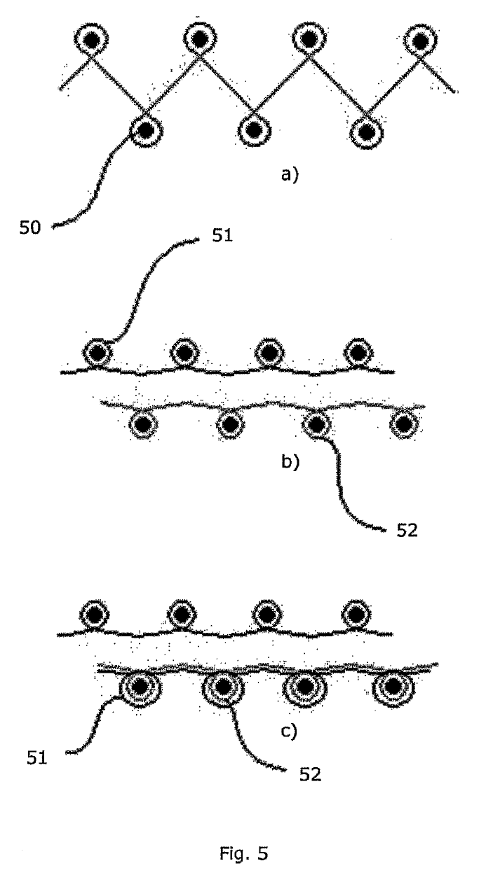

The whole fabric is made by using a double needle-bed, as shown in FIG. 5 (a-c), where black points 50 represent needles, the dark line 51 represents the ground yarn and the light line 52 represents the metal yarn.

As shown in FIG. 5 (a), the jersey is made with a double-needle bed showing the same knitting on the face side and the reverse sides of the fabric; the favourite configuration is a jersey knitted structure done with a double bed machine.

In the intarsia regions, corresponding to electrodes, FIG. 5 (b), metal yarns 52 are located only on the reverse side of fabric, corresponding to the side in contact with the skin, and the two needle-beds are running separately and a layers of the knitted fabric made with the ground yarn 51 insulates the electrode from the exterior. The use of a double-bed machine allows the separation of the series of needles, so that, during the production of the fabric, two separate layers are obtained: one external, made of ground yarn, which insulates the internal layer, made of metal yarn, from the exterior. This technique is known as tubular knitting.

Finally, as shown in FIG. 5 (c), to obtain connections a further layer of knitted fabric is needed: by means of Vanise technique, it is possible to use two different yarns 51 and 52 contemporaneously on the same series of needles, overlapping the ground yarn 51 to the metal one 52. With this technique, a layer of fabric can be obtained where each yarn is visible only on one side. With this process the metal yarn in the final fabric structure is insulated from the body of the user. At the same time, the second series of needles (as a double-bed machine is used) is knitting a ground-based layer which insulate the metal layers from exterior.

Electrodes are made of metal yarns, specifically copper and steel, twisted with standard yarns; the same metal yarns are used for the production of connections. Piezoresistive yarns are elastic yarns composed by electro-conductive fibres (like metal fibres, e.g. stainless steel, or electro-deposited fibres), or synthetic fibres (like polyamides), containing dispersed phases or shells of conductive materials, like e.g. carbon.

Signals detected by sensors are transferred, by the way of integrated and insulated connections, to a miniaturised, portable, electronic device that cares for filtering, elaboration, storage and transmission of detected data (see FIG. 3). At this point signals are acquired, elaborated and transmitted to a remote system, where they are correlated so that a series of indexes are extracted to generate alert signals. A related feedback signal can be sent back to the user, helping the user to integrate this information with personal feeling on his/her health perception, enhancing personal self-management and reaction to potential health risks. In case feedback information could be of danger for the patient, e.g. it could generate panic, an enhanced system can alert physicians instead of the patient, so that the crisis can be managed by specialised personnel.

The knitted fabric above described shows the characteristics of wearability and comfort necessary for the implementation of a system able to monitor the user or the patient during his/her normal activity, or in case of risks, without preventing, reducing or modifying patient behaviour and without preventing monitoring. This allows a higher quality of life for patients, who can be monitored remotely, and to acquire information in real life, and not only during controlled conditions (e.g. hospitals, physician laboratories).

The present invention can be exploited as a prevention and early diagnosis system and can be used to help people understanding physiological signals and improve awareness of their organism.

Type and number of sensors depend on applications; for cardiopath patients the number of leads and electrodes is higher than at-risk workers, as for the latter is more important the detection of other parameters like respiration, movement activity, posture, etc.

As it is fundamental that the system is in tight contact with the user's body, so that artefacts due to movement are reduced, the fabric is preferably elastic. The system can be disguised as a normal garment, helping the patient to conceal the use of a monitoring device.

Sensors and electrodes location, as well as connection network, is the result of a morphologic study, aimed to increase a signal-to-noise ratio. The preferred configuration is shown in FIGS. 1 and 2.

Finally, piezoresistive sensors are realised with the same intarsia technique. A more sensitive piezoresistive sensor to be used for the respiratory signal can be made with a lycra-based fabric coated with carbon-loaded rubber or latex: a piece of this fabric embedded inside a more rigid band, can be hidden in tubular structures at thorax and abdominal level.

To increase elasticity of the garment, the whole fabric is made knitting an elastic yarn together with the other yarns.

The present invention includes both cut-and-sewn clothes, where pieces of fabric using the above described techniques are sewn together, and seamless clothes, ready to be worn. In the former case, it is necessary to develop a pattern corresponding to final garment. The final garment is manufactured by cutting different pieces of knitted fabric by means of patterns and sewing them in the final shape.

Sleeves are manufactured to ensure the higher sensitivity of movement sensors, by cutting the sleeve shape from the fabric rotated with respect to knitting direction, so that course in the sleeve are parallel to arm length (“appiombo”).

Results obtained with embedded electrodes have been validated comparing the signals with those obtained simultaneously with standard Ag/AgCl electrodes, positioned on lead V1 (D1 for Eindhoven leads) of ECG.

Electrodes are coupled with a disposable Hydrogel™ membrane.

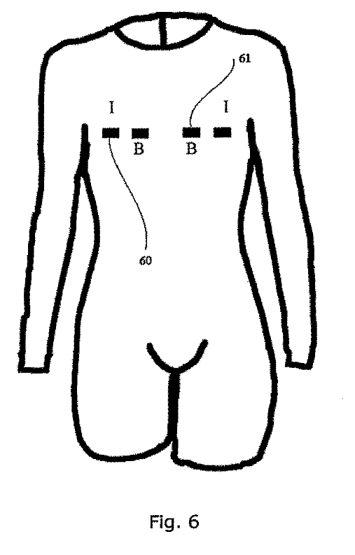

An alternative method to measure respiratory activity is the use of the impedance pneumography. In this case four electrodes are placed on thoracic position, as shown in FIG. 6.

The two external ones 60 are used to inject a high frequency current (50 kHz) and the other ones 61 allow capturing the voltage variation caused by thoracic impedance change. The output signal is modulated by changes in the body impedance accompanying the respiratory cycle. The change in impedance corresponding to each respiratory cycle is of the order of 1-4% of the base impedance. The relationship between impedance change ΔZ and volume of air moved (ΔV) is approximately linear under most circumstances. Since the impedance changes is related to the volume of air moved, the method can be calibrated.

1-18. (canceled)

19. Knitted fabric wherein piezoresistive sensors for the monitoring of movement and breathing, electrodes for the monitoring of cardiac activity and breathing, and conductive connections for the transmission of signals are integrated, wherein said knitted fabric is made of multiple layers where sensors, electrodes and connections are located.

20. Knitted fabric according to claim 19 wherein said piezoresistive sensors are realized by regions of fabric made of piezoresistive yarns.

21. Knitted fabric according to claim 20 wherein said piezoresistive sensors are realized by the so-called “intarsia” technique.

22. Knitted fabric according to claim 19 wherein said electrodes and said conductive connections are realized by conductive yarns.

23. Knitted fabric according to claim.22 wherein said electrodes and said conductive connections are made using a tubular intarsia technique.

24. Knitted fabric according to claim 19 wherein said electrodes are made of metal yarns twisted with standard yarns.

25. Knitted fabric according to claim 20 wherein said piezoresistive yarns are elastic yarns composed by electro-conductive fibers or synthetic fibers containing dispersed phases or shells of conductive materials.

26. Knitted fabric according to claim 20 wherein said piezoresistive yarns are made with a lycra-based fabric coated with carbon loaded rubber.

27. Knitted fabric according to claim 19 wherein said conductive connections are made of metal yarns twisted with standard yarns.

28. Knitted fabric according to claim 19 wherein said knitted fabric is made using the double-bed jersey technique.

29. Knitted fabric according to claim 19 wherein said knitted fabric is made of multiple layers in a way that electrodes are placed in contact with the skin of the user under examination while connections are insulated by a layer of fabric which separates them from the user's body.

30. Knitted fabric according to claim 19 wherein said piezoresistive sensors and said electrodes are located as reported on FIG. 1.

31. Knitted fabric according to claim 19 wherein said electrodes are located as reported on FIG. 6.

32. Knitted fabric according to claim 19 wherein said knitted fabric is employed in cut and sewn clothes and garments.

33. Knitted fabric according to claim 31 wherein the sleeves comprised in said clothes and garments have a shape that is cut from said fabric, rotated with respect to knitting direction so that course in said sleeves are parallel to arm length.

34. A method for the detection of signals related to ECG, EOG, EMG, respiratory activity or respiratory frequency, said method comprises:
using a knitted fabric wherein piezoresistive sensors for the monitoring of movement and breathing, electrodes for the monitoring of cardiac activity and breathing, and conductive connections for the transmission of signals are integrated, wherein said knitted fabric is made of multiple layers where sensors, electrodes and connections are located.

35. A method for the detection of signals related to movement activity, said method comprising:
using a knitted fabric wherein piezoresistive sensors for the monitoring of movement and breathing, electrodes for the monitoring of cardiac activity and breathing, and conductive connections for the transmission of signals are integrated, wherein said knitted fabric is made of multiple layers where sensors, electrodes and connections are located.

36. A method for the detection of impedance pneumography, said method comprising:
using a knitted fabric wherein piezoresistive sensors for the monitoring of movement and breathing, electrodes for the monitoring of cardiac activity and breathing, and conductive connections for the transmission of signals are integrated, wherein said knitted fabric is made of multiple layers where sensors, electrodes and connections are located.

37. Process for the production of a knitted fabric wherein piezoresistive sensors for the monitoring of movement and breathing, electrodes for the monitoring of cardiac activity and breathing, and conductive connections for the transmission of signals are integrated, wherein said knitted fabric is made of multiple layers where sensors, electrodes and connections are located, wherein said knitted fabric is made using the double-bed jersey technique.

38. Process according to claim 37 wherein said electrodes and said conductive connections are made using the so-called “tubular intarsia technique”.

39. Process according to claim 38 wherein said knitted fabric is made with double bed weft knitting machines.
